(19) United States
(12) Patent Application Publication
(10) Pub. No.: US 2017/0360863 A1
(43) Pub. Date: Dec. 21, 2017

(54) USE OF POLYACETYLENIC GLYCOSIDES FOR SUPPRESSION OF GRANULOCYTIC MYELOID-DERIVED SUPPRESSOR CELL ACTIVITIES AND TUMOR METASTASIS

(71) Applicant: Academia Sinica, Taipei City (TW)

(72) Inventors: Ning-Sun YANG, Portland, OR (US); Wen-Chi WEI, Taipei (TW); Sheng-Yen LIN, Taipei (TW); Pei-Wen HSIAO, Taipei (TW); Yet-Ren CHEN, Taipei (TW)

(21) Appl. No.: 15/534,089

(22) PCT Filed: Dec. 9, 2015
(86) PCT No.: PCT/US15/64841

§ 371 Date: Jun. 8, 2017

Related U.S. Application Data
(60) Provisional application No. 62/091,474, filed on Dec. 12, 2014.

(51) Int. Cl. A61K 36/28; A61K 39/395; C07K 16/28; A61K 39/00; A61K 45/06

(52) U.S. Cl. CPC A61K 36/28; A61K 39/39558; C07K 16/2803; A61K 45/06; A61K 2039/505; C07K 2317/92; A61K 2236/00

(57) ABSTRACT

A pharmacological composition for use in inhibiting differentiation, functional activities, and population of granulo-cytic myeloid-derived suppressor cells (gMDSCs) and/or suppressing, tumor metastasis in a subject in need thereof is disclosed. The composition comprises a therapeutically effective amount of Bidens pilosa extract, or more than one polyacetylenic compounds purified or isolated from the B. pilosa extract, and a pharmaceutically acceptable carrier.

### FIELD OF THE INVENTION

The present invention relates generally to methods for suppressing myeloid-derived suppressor cells.

### BACKGROUND OF THE INVENTION

Owing to the recent advancement in precision surgeries, early diagnosis of cancer, and adjuvant therapies with chemotherapeutic drugs, cancer death rate now is mainly reflecting the degree and pattern of residual or circulating tumor cells metastasizing from the primary tumor site to the secondary tissue target sites. Initiation of metastatic process was evidenced in 60% to 70% of patients by the time of diagnosis or thereafter. Control, blockage and prevention of such metastasis have hence been recognized as the key steps for successful intervention with cancer metastasis. Currently, therapy for metastatic disease still encounters great challenges.

Myeloid-derived suppressor cells (MDSCs) are main immunosuppressive cells that have been shown to negatively regulate immune responses against cancers. MDSCs are shown to be largely responsible for inhibiting host antitumor immunities and consequently impairing the effectiveness of anticancer immunosuppressive therapeutic approaches, MDSCs are a heterogeneous population of cells that consists of myeloid progenitor cells and immature myeloid cells (IMCs) present during tumor progression, tissue inflammation and pathogen infection. Two different subtypes of MDSCs, namely monocytic MDSCs and granulocytic MDSCs (mMDSCs and gMDSCs, respectively), have been identified based on their morphology, biomarkers and functions. Various MDSCs have therefore been recognized to play a hierarchical role in tumor-induced immunosuppression activity. As a result, the strategy of preventing or blocking the development of MDSCs in cancer patients is being considered as a prime approach for cancerous diseases.

### SUMMARY OF THE INVENTION

In one aspect, the invention relates to a pharmacological composition comprising: (i) a therapeutically effective amount of Bidens pilosa extract or more than one polyacetylenic compounds purified or isolated from the B. pilosa extract; and (ii) a pharmaceutically acceptable carrier, for use in suppressing, blocking and/or preventing tumor metastasis in a subject in need thereof.

Alternatively, the invention relates to use of the aforementioned pharmacological composition in the manufacture of a medicament for suppressing, reducing, blocking and/or preventing tumor metastasis in a subject in need thereof.

The invention also relates to a method for suppressing, blocking and/or preventing tumor metastasis in a subject in need thereof, comprising administering to the subject in need thereof the aforementioned pharmacological composition.

In another aspect, the invention relates to a pharmacological composition comprising: (i) a therapeutically effective amount of Bidens pilosa extract, or more than one polyacetylenic compounds purified or isolated from the B. pilosa extract; and (ii) a pharmaceutically acceptable carrier, for use in inhibiting differentiation, functional activities, and population of granulocytic myeloid-derived suppressor cells (gMDSCs) and/or suppressing metastatic cancer or cancer metastasis in a subject in need thereof.

Alternatively, the invention relates to use of the aforementioned pharmacological composition in the manufacture of a medicament for inhibiting differentiation, functional activities, and population of granulocytic myeloid-derived suppressor ceils (gMDSCs) and/or suppressing metastatic cancer or cancer metastasis in a subject in need thereof.

The invention relates to a method for inhibiting differentiation, functional activities, and population of granulocytic myeloid-derived suppressor cells (gMDSCs) and/or suppressing metastatic cancer or cancer metastasis in a subject in need thereof, comprising: administering to the subject in need thereof the aforementioned pharmacological composition.

In another embodiment of the invention, the pharmacological composition comprises at least 80% or no less than 89% (wt/wt) of compounds 2-β-D-glucopyranosyloxy-1-5(E)-tridecene-7,9,11-triyne, 2-D-glucopyranosyloxy-1-hydroxytrideca-5,7,9,11-tetrayne, and 3-β-D-glucopyranosyloxy-1-hydroxy-6(E)-tetradecene-8,10,12-triyne.

In another embodiment of the invention, the pharmacological composition comprises: (a) 2-β-D-glucopyranosyloxy-1-hydroxy-5(E)-tridecene-7,9,11-triyne, (b) 2-D-glucopyranosyloxy-1-hydroxytrideca-5,7,9,11 -tetrayne, and (c) 3-β-D-glucopyranosyloxy-1hydroxy-6(E)-tetradecene-8,10,12-triyne at a ratio ranging from 1:1:2 to 1:2:4, or from 1:1:1 to 1:2:4.

In another embodiment of the invention, the subject has breast cancer, or is a post-operative cancer surgery patient, or in need for control, blockage and prevention of cancer metastasis.

In another embodiment of the invention, the pharmaceutical composition inhibits differentiation, functional activities, and population of granulocytic myeloid-derived suppressor ceils (gMDSCs) and suppresses tumor metastasis without causing cytotoxicity or apoptosis to the gMDSCs.

In another embodiment of the invention, the pharmaceutical composition is in a dosage form selected from the group consisting of oral, intravenous, intramuscular, and subcutaneous.

In another embodiment of the invention, the amount of the Bidens pilosa extract or the more than one polyacetylenic compounds purified or isolated from the B. pilosa extract is effective in inhibiting

tumor metastasis into lung, and accumulation of granulocytic MDSCs in lung, peripheral blood and spleen of the subject in need thereof.

In another embodiment of the invention, the Bidens pilosa extract is: (i) an ethanol extract of B. pilosa; or (ii) a first fraction eluted from an HPLC column loaded with a mixture containing the ethanol extract of B. pilosa, or (iii) a repeatedly re-chromatographed fraction of the ethanol extract of B. pilosa.

In another embodiment of the invention, the B. pilosa extract comprises no less than 89% (w/w) of polyacetylenic compounds.

In another embodiment of the invention, the pharmaceutical composition comprises a human equivalent dose of: (a) 10-1000 mg of the ethanol extract of B. pilosa/Kg body weight×(0.025 Kg/human body weight in Kg)^{0.33}, or (b) 0.5-1000 mg of the first fraction/Kg body weight×(0.025 Kg/human body weight in Kg)^{0.33}.

In one embodiment of the invention, the pharmacological composition comprises compounds of formula (I), (II) and (III):

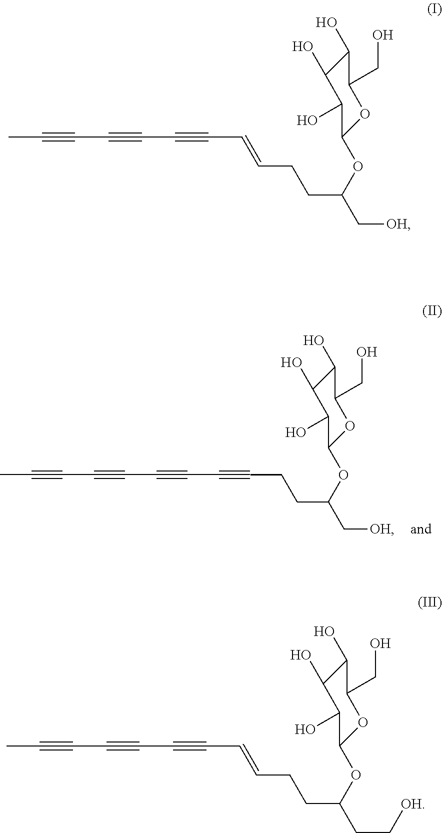 [SMILES: CC#CC#CC#C/C=C/CCC(CO)OC1OC(CO)C(O)C(O)C1O ; CC#CC#CC#CC#CCCC(CO)OC1OC(CO)C(O)C(O)C1O ; "and" ; CC#CC#CC#C/C=C/CCC(CCO)OC1OC(CO)C(O)C(O)C1O]

These and other aspects will become apparent from the following description of the preferred embodiment taken in conjunction with the following drawings. The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIGS. 1A-D show change of myeloid derived suppressor cell populations and G-CSF level in blood and spleen tissues murine 4T1 tumor-bearing mice. Test mice were implanted orthotopically with 5×10⁵ 4T1-luc2 cells and monitored weekly by non-invasive bioluminescent imaging. (A) Representative weekly bioluminescent imaging of tumor-bearing mice. (B) Quantitation of bioluminescent imaging (BL1) of test tumors (A) (Black bar) and expression of serum G-CSF level (White bar) in tumor-bearing mice. (C) Population distribution of gMDSCs and mMDSCs in blood cells (Dark line) and splenocytes (Spotted line) in tumor-bearing mice, analyzed by flow cytometry. (D) Weight of tumor mass (Dark line) and spleen (Spotted line) in tumor-bearing mice.

FIGS. 2A-E show correlation between expression levels of gMDSCs, G-CSF and the rate of tumor growth and metastasis. Test mice were implanted orthotopically with 5×10⁵ 4T1-luc2 cells and primary tumors were resected at Day 21 post tumor implantation. (A) Quantitative data of bioluminescent imaging (BLI, Black bar) and serum G-CSF level (White bar) in tumor-resected mice, scored between Day 7 and Day 35. (B) Correlation between population frequency of gMDSCs and serum G-CSF Level in tumor-resected mice. (C) Correlation between survival time (day) and serum G-CSF level. (D) Test mice were co-injected orthotopically with 4T1 cells (5×10⁵) and granulocytic MDSCs and primary tumors were resected at Day 18 post tumor implantation. Tumor mass of two test groups are shown. (E) The incidence of free from metastasis in mice treated with 4T1 only (solid circle) versus 4T1 plus MDSC (solid square) is presented.

FIGS. 3A-E show the effect of the ethanol-extracted fraction of Bidens pilosa (BP-E) on the functional and differentiation activities of MDSCs and on G-CSF expression. (A) Population of granulocytic MDSCs in treated bone marrow cells were determined by flow cytometry. (B) Cytotoxicity of BP-E on bone marrow cells, revealed by MTT assay at 24 hours post treatment (C) Expression of G-CSP receptor in BP-E treated 4T1 cells, shown by Western blotting analysis. (D) Cells were treated at serial concentrations (12.5 to 100 μg/mL) of BP-E for 24 hours and ROS expression in MDSCs were measured by incubating cells with H₂DCFDA fluorescent probes. (E) Ex vivo cytotoxicity of BP-E on bone marrow cells, revealed by MTT assay for 24 hours.

FIGS. 4A-E show the effect of ethanol-fractionated phytochemicals from Bidens pilosa (BP-E) on tumor metastasis. (A) Tumor volume of untreated and BP-E treated mice was shown. (B) Bioluminescent imaging from untreated and BP-E treated mice at 7 days post tumor resection. (C) The incidence of free from metastasis in control and BP-E treated group mice. (D) Survival rate of test mice. (E) Weight of spleen tissue in test mice on day 21 post tumor resection was shown.

FIGS. 5A-D show the effect of the F1 fraction of BP-E (BP-E-F1) on ROS expression in MDSCs and on differentiation of MDSCs from bone marrow cells. (A) HPLC profiling with an absorbance of UV 235 nm of BP-E separated into 4 major sub-fractions (F1, F2, F3, and F4). (B) Cell number of MDSCs differentiated from bone marrow cells in treated cells was determined by flow cytometry analysis. (C) Population of granulocytic MDSCs differentiated from bone marrow cells in treated cells was determined by flow cytometry analysis. (D) Cells were treated with four sub-fractions (F1, F2, F3, and F4) at 10 μg/mL for 24 hours and ROS expression in MDSCs were measured by incubating cells with H₂DCFDA fluorescent probes.

FIGS. 6A-B show the results of chemical identification of F1 phytochemicals. (A) Chromatograph of F1 fraction by a RP-18 UPLC column. (B) Chemical structure of 3 major compounds (2-β-D-glucopyranosyloxy-1 -hydroxy-5(E)-tridecene-7,9,11 -triyne, 2-D-glucopyranosyloxy-1-hydroxytrideca-5,7,9,11 -tetrayne, and 3-β-D-glucopyranosyloxy-1-hydroxy-6(E)-tetradecene-8,10,12-triyne) in F1 identified by spectroscopic methods.

FIGS. 7A-G show the effect of BP-E-F1 on tumor metastasis. (A) Tumor volume of control and BP-E-F1 group mice was shown. (B) Bioluminescent images of all test mice at 23 days post tumor resection were shown. (C) Quantitative data of bioluminescent images in whole body of all test mice. (D) The incidence of free from metastasis in control BP-E-F1, and Docetaxol treated mice. (E) Body weight of all test mice. (F) Representative bioluminescent images of liver, lung, and spleen in test mice at 23 days post tumor resection were shown. (G) Population of granulocytic and monocytic MDSCs in preferred organs of test mice was determined by flow cytometry.

FIGS. 8A-D show that BP-E-F1 inhibits MDSC activities on tumor growth and metastasis. (A) Tumor volume of control BP-E-F1, and BP-E-F1+MDSCs group mice, (B) Tumor weight of all test groups at 18 days post tumor implantation. (C) The incidence of free from metastasis in all test groups. (D) Bioluminescent imaging of all test groups at 14 days post tumor resection.

FIGS. 9A-B show the results of pharmacokinetic study of F1 fraction. (A) The concentrations of the three compounds (A-C) of F1 fraction in test sera were determined by liquid chromatography-tandem mass spectrometry (LC/MS/MS). The absolute bioavailability of oral administration is then determined by the dose-corrected area under curve (AUC) of oral administration divided by AUC of iv administration. (B) The tissues of bone, kidney, lung, liver and spleen in BP-E-F1 treated mice were collected and the concentrations of the three compounds (A, B, and C) were detected by liquid chromatography-tandem mass spectrometry (LC-MS/MS).

FIGS. 10A-C show that F1 fraction inhibits G-CSF-induced granulocyte differentiation and signaling transduction. (A) Cell number of granulocytes in peripheral blood of test mice was determined by using a hematology analyzer. (B) Expression of phosphorylation of STAT3 and total STAT3 in representative bone marrow cells in vivo were measured by western blotting analysis. (C) Expression of phosphorylation of STAT3 and total STAT3 in treated gMDSCs ex vivo were determined by western blotting analysis.

### DETAILED DESCRIPTION OF THE INVENTION

### Unique Features and Advantages of the Invention When Compared to the Existing Technologies

Growing body of evidence suggests now that chemotherapy, performed as a systemic therapy for metastatic cancer, does not benefit to all cancer patients, but impairs host immunity resulting in the promotion of tumor growth and spread. The invention relates to the discovery that oral administration of BP-E or F1 fraction of BP-E significantly suppressed metastasis. The efficacy of F1 fraction in inhibition of metastasis and MDSC accumulation was as good as docetaxel treatment. Moreover, Mice fed F1 fraction showed better general health than docetaxel-treated mice. F1 fraction, unlike docetaxel, did not induce body weight loss or hair loss in our murine mammary tumor resection model.

### Commercial Applications of the Invention

Comparing the efficacy, drug administration and side effects of F1 fraction and the current clinical drag docetaxel, this invention is based on an unexpected discovery that phytochemicals prepared from B. pilosa (including BP-E and F1 fractions) can suppress differentiation and functions of MDSC and inhibit mammary tumor metastasis. These extracts can be used as anti-cancer agent against MDSC and tumor metastasis of breast cancers.

As used in the description herein and throughout the claims that follow, the meaning of “a”, “an” and “the” includes plural reference unless the context clearly dictates otherwise. Also, as used in the description herein and throughout the claims that follow, the meaning of “in” includes “in” and “on” unless the context clearly dictates otherwise.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description, of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only.

Unless otherwise defined, all technical and scientific terms used herein have the same meaning as commonly understood by one of ordinary skill in the art to which this invention pertains. In the case of conflict, the present document, including definitions will control

The term “treating” or “treatment” refers to administration of an effective amount of a therapeutic agent to a subject in need thereof, who has a disease (such as tumor and/or tumor metastasis), or a symptom or predisposition toward such a disease, with the purpose of cure, alleviate, relieve, remedy, ameliorate, or prevent the disease, the symptoms of it, or the predisposition towards it, or reduce incidence of symptoms. Such a subject can be identified by a health care professional based on results from any suitable diagnostic method.

“An effective amount” refers to the amount of an active compound that is required to confer a therapeutic effect on the treated subject. Effective doses will vary, as recognized by those skilled in the art, depending on route of administration, excipient usage, and the possibility of co-usage with other therapeutic treatment.

The terms “ethanol extract of B. pilosa” and “BP-E phytoextract” are interchangeable. An ethanol extract of B. pilosa refers to “the phytochemicals extracted from fresh or dried tissues of whole plant of Bidens pilosa Linn var. radiata (Asteraceae) by using ethanol (e.g., 95% EtOH)”.

The terms “F1 fraction” refers to “BP-E derived F1 phytochemicals”. The “F1 fraction” is a sub-fraction of BP-E phytoextract which was isolated by fractionation with HPLC. For example, using a PR-18 preparative HPLC column (e.g., COSMOSIL™ C18, 4.6 mm×250 mm) and a UV 235 nm detector, and a MeOH/H₂O gradient at a flow rate of 0.5 ml/min, the elute fraction was collected at the retention time of 40 min to 46 min.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

As used herein, when a number or a range is recited, ordinary skill in the art understand it intends to encompass an appropriate, reasonable range for the particular field related to the invention.

By 0.5-1000 mg it meant that all tenth and integer unit amounts within the range are specifically disclosed as part of the invention. Thus, 0.5, 0.6, 0.7 and 1, 2, 3, 4 . . . 999.7, 999.8, 999.9 and 1000 unit amounts are included as embodiments of this invention.

The current study investigated the immune-regulatory and antitumor activities of the ethanol extract of B. pilosa (BP-E) on MDSC expansion and tumor metastasis. The results show that BP-E can effectively suppress the metastasis of 4T1 tumors and increase animal survival in a mouse mammary tumor-resection model. BP-E significantly decreased the tumor-induced splenomegaly and, mechanically, it specifically inhibited the differentiation and functional activities of granulocytic MDSCs and reduced the population of these cells in test mice. Bio-organic chemistry-analysis shows that specific polyacetylenic glycosides from the F1 fraction of BP-E are the major principle phytochemicals responsible for the detected MDSC and anti-metastatic activities. Our findings suggest that specific polyacetylene compounds from B. pilosa be readily and highly purified or F1 fraction and they may have useful application for future development as botanical drug(s).

It was discovered that high level expressions of G-CSF and gMDSC populations were detected with a pattern of different stages of a murine 4T1 mammary carcinoma model in tumor-bearing mice. The ethanol extract of B. pilosa (BP-E) exhibited a strong immunomodulatory capacity that can effectively suppress the G-CSF-induced differentiation of gMDSCs from bone marrow cells ex vivo, and can suppress with high potency 4T1 tumor metastasis in a tumor-resection model. The ethanol extract of B. pilosa (BP-E) can effectively suppress metastasis and increase animal survival in a mouse mammary tumor-resection model BP-E significantly decreased tumor-induced splenomegaly and, mechanically, it specifically inhibited the differentiation and functional activities of granulocytic MDSCs and reduced the population of these cells in test mice.

We further demonstrated that oral delivery of BP-E can suppress tumor metastasis via inhibiting the differentiation and function of gMDSCs in test mice. Bio-organic chemistry analysts showed that a specific group of polyacetylenic glycosides, as the great majority constituents (≧89%) of the F1 fraction of BP-E, apparently act as active phytochemicals responsible for the effect on MDSC activities ex vivo and in vivo, and the resultant anti-metastatic activities in vivo. This indicates that phytochemicals in BP plant extracts or the derived ethanol fraction may have therapeutic or other clinical applications.

### EXAMPLES

Exemplary instruments, apparatus, methods and their related results according to the embodiments of the present invention are given below.

### Materials and Methods

### Extraction of Plant Tissues, Compound Isolation and Identification

Bidens pilosa Linn. Var radiate (Asteracear) plants were grown in farms of Sanxia district, New Taipei city, Taiwan, in 2013. Air-dried shoot, leaf and root tissues of whole plants, weighting 228.2 g, were imbibed, extracted in 2.28 liters of 95% ethanol (EtOH) at room temperature for three days. This total crude extract was evaporated in vacuum to yield a dried residue (6.3955 g), that then resuspended in methanol (MeOH) and eluted with a water-MeOH mixture of decreasing polarity using a PR-18 preparative HPLC column [COSMOSIL™ C18, 4.6 mm×250 mm] with a flow rate of 0.5 ml/mm and detected at UV 235 nm to give a total of 4 sub-fractions (F1-F4). F1 (eluent of 73.5% MeOH/water from the PR-18 column) was collected at the retention time of 40 min to 46 min and identified as a bioactive fraction. The F1 was also repeatedly separated by the same eluted with 70% to 72% MeOH in water for further using in vitro and in vivo.

F1 was subsequently chromatographed by a RP-18 UPLC column [Acquity UPLC HSS C-18 column 2.1×150 mm, 1.8 um] eluted with 30% to 32% Acetonitrile (ACN) with 0.2% Trifluoroacetic acid (TFA) to give a total of four 2^{nd} sub-fractions, FF. A-FF. D. These 2^{nd} sub-fractions were further separated from Fr.1 (40 mg) by a PR-18 preparative HPLC column [COSMOSIL™ CIS, 10 mm×250 mm] eluted with 31.2% ACN with 0.05% TFA to afford compound A (FF. A. 7 mg), compound B (FF. B, 10 mg), and compound C (FF. C+D, 18.79 mg). Those structures, 2-β-D-glucopyranosyloxy-1-hydroxy-5(E)-tridecene-7,9,11-triyne (A), 2-D-glucopyranosyloxy-1-hydroxytrideca-5,7,9,11-tetrayne (B) and 3-β-D-glucopyranosyloxy-1-hydroxy-6(E)-tetradecene-8,10,12-triyne (C), were compared and confirmed by their NMR and MS/MS data.

### Animal Studies

4T1-luc2 cells (5×10⁵ cells/100 μl PBS) were orthotopically implanted into mammary fat-pad of BALB/c mice. Primary tumor growth was evaluated by measuring tumor weight and by monitoring bioluminescent imaging of mammary tumors (BL1) every 7 days. For tumor resection mouse model, 4T1-luc2 cells (5×10⁵ cells/100 μl PBS) were orthotopically implanted into mammary fat pad of test mice. At day 21 post tumor implantation, the tumor mass was gently surgically removed. Bioluminescent imaging of metastatic tumor was monitored by using Non-invasive in vivo imaging system (IVIS). The body weight of the test mouse was approximately 25 g.

Construction of 4T1-luc2 Cells

293T cells were transfected with pMD.G, pCMV ΔR8.91, and pIF4g.As2.luc.bla to construct lentivirus with luc2 gene. After 24 hour, cell medium were collected and added to transfect 4T1 cells with constructed virus. 10 μg/mL blasticidin S was applied to select the single clone of 4T1-luc2 cells. 4T1-luc2 cells were cultured and maintained in RPMI-1640 supplemented with 10 μg/mL Blasticidin S, 10% fetal bovine serum, 1 mM Penicillin/Streptomycin, and 1 mM sodium pyruvate at 37° C. in 5% CO2 and 95% humidity.

### Cell Population Analysis

Lung tissues of test mice were harvested and minced with 150 U/mL Type I Collagenase in tissue grinders for 20 to 50 times. After digestion and lysed with ACK buffer, grinded tissues were collected and filtered through 40 μm cell strainer. Spleen tissues were minced gently with PBS in tissue grinders. After lysed with ACK buffer, cells were collected for further analysis. Blood were lysed with ACK buffer for 3 times and harvested for further analysis. All cells were collected and stained with anti-CD11b, and anti-Ly6G/Ly-6C for flow cytometry analysis.

gMDSCs Isolation

To purify Ly-6G⁺ MDSCs, splenocytes of tumor-bearing mice were harvested and depleted erythrocytes by ACK buffer. Then, splenocytes were incubated with anti-Ly-6G-biotin Abs for 20 minutes and followed by positive selection using anti-biotin microbeads, following the manufacturer's instructions (MiltenyiBiotec).

### Bone Marrow Cells Preparation

BALB/c mice bone marrow cells from femorae and tibiae were depleted of RBCs with ACK lysis buffer and cultured in RPMI 1640 medium supplemented with 20 ng/ml GM-CSF, 10% fetal bovine serum, 50 μM 2-mercaptoethanol, 100 unit/ml penicillin and 100 μg/ml streptomycin in a humidified 5% CO₂ incubator at 37° C.

### Immunoblotting

Cells lysates were prepared by using M-PER Mammalian Protein Extraction Reagent [5 mM bicine buffer, 4-(2-aminoethyl)benzenesulfonyl fluoride (AEBSF 0.3 mM), leupeptin (10 μg/ml) and aprotonin (2 μg/ml)]. Lysates were run on 5% to 20% gradient polvacrylamide-sodium dodecyl sulphate (SDS) gels (20 μg protein per lane), proteins transferred onto Hybond-ECL membranes (GE-Healthcare; Amersham, UK) and immunoblotted with anti-G-CSFR antibody, anti-stat3 antibody, and anti-phosphorylated stat3 antibody. Protein bands were detected by enhanced chemiluminescence (Clarity Western ECL Substrate, BioRad) and developed by autoradiography.

### Detection of Serum G-CSF by ELISA

Serum from test mice and conditional medium were collected and stored at −80° C. until assayed. Samples were checked for expression levels of G-CSF (R&D Systems) and quantified at a wavelength of 450 nm using a BiotekPowerWave HT spectrophotometer.

### Antibodies

Anti-stat3 antibody and anti-phosphorylated stat3 antibody were purchased from Cell Signaling Technology. Anti-G-CSFR antibody was purchased from Abcam.

### Statistical Analysis

Data are presented in fold changes or in percentages with mean±s.e.m. indicated in figure legends. All statistical analyses were determined using GraphPad Software. As comparison between multiple data sets, a one-way ANOVA analysis with Tukey-Kramer method was performed.

### Results

### Change of Myeloid Derived Suppressor Cell Populations and G-CSF Level in Blood and Spleen Tissues Murine 4T1 Tumor-bearing Mice

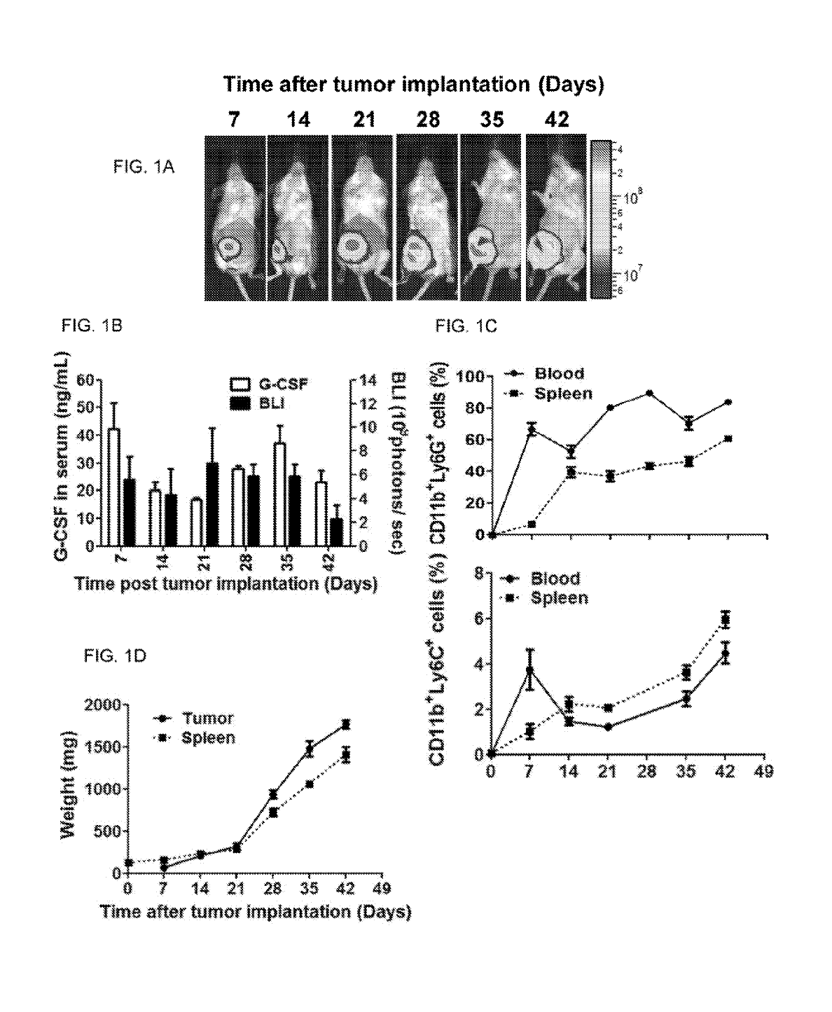

MDSCs have been shown to expand in cell population in cancer patients. Granulocyte colony-stimulating factor (G-CSF) was shown as one of the key cytokines secreted by tumor cells that mediate MDSC production. To characterize the dynamic change of MDSC population and G-CSF expression in 4T1 tumor-bearing mice, transgenic 4T1luc2 cells were orthotopically implanted into mammary fat pad of test mice. The representative bioluminescent imaging on growth of orthotopic 4T1-luc2 tumor was recorded weekly (FIG. 1A). The levels of bioluminescence intensity (BLI) and G-CSF in tumors of test mice was determined. High level of BLI was detected in mice from day 7 to day 42 post tumor implantation (i.e., BLI>2×10⁹ photons/sec) in accordance to a time course pattern as that observed for expression of G-CSP in test mice (FIG. 1B). The population of gMDSCs expressing CD11b⁺Ly6G⁺ in white blood cells (WBCs) of peripheral blood reached 66.7% at day 7, and maintained at a high level (89% to 52% of total WBCs) from day 14 to day 42 (FIG. 1 C). High level gMDSCs (≧35% of splenocytes) in the spleen of test mice was detected from day 14 to day 42 (FIG. 1C). Monocytic MDSCs (mMDSCs) expressing CD11b⁺Ly6C⁺ in WBCs of peripheral blood and in spleen tissue were found between 1%˜6% (FIG. 1C). In addition, the weight of tumor and spleen tissue in test mice were gradually increased between day 7 to 21, but dramatically increased at day 21 post tumor implantation (FIG. 1D).

Correlation Between Expression Levels of gMDSCs, G-CSF and the Rate of Tumor Growth and Metastasis

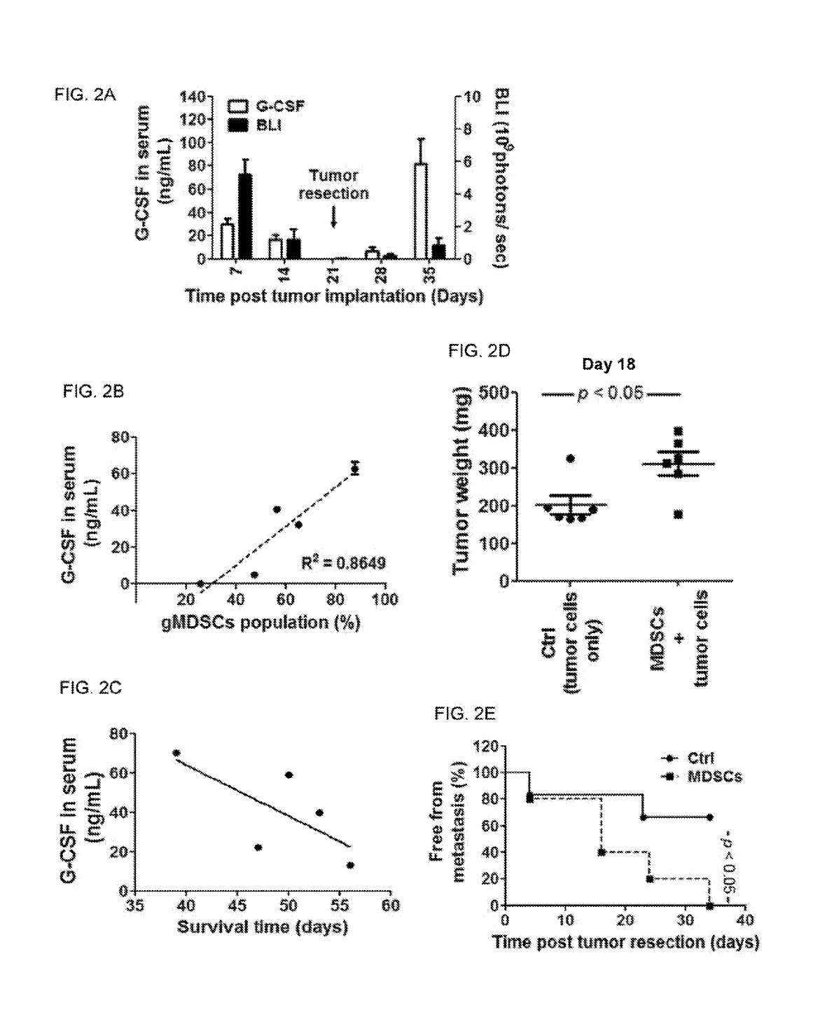

Expression levels of gMDSCs and G-CSF were previously shown to be closely associated with the progression of tumor growth in mouse models. To investigate the role of gMDSCs and G-CSF in growth and metastasis of mouse mammary tumors, we orthotopically implanted 4T1-luc2 cells into mammary fat pad of test mice. At day 21 post tumor implantation, the primary tumor mass was gently removed surgically. The level of tumor bioluminescence intensity and expression of G-CSF were weekly measured (FIG. 2A). Expression level of G-CSF in serum of test mice at day 21 was dramatically reduced soon after tumor resection, indicating that the high level G-CSF detected in circulation of 4T1 tumor-bearing mice was mainly secreted by cells of the tumor mass (FIG. 2A). After tumor resection, test mice with metastatic tumor(s) have gradually resumed the high level expression of G-CSF in blood serum. This pattern of G-CSF expression in test mice was well correlated with the population increase of granulocytic MDSCs (FIG. 2B) and the increase in G-CSF level in test mice was inversely correlated with mouse survival time after tumor resection (FIGS. 2B-C). These results suggeste that gMDSC population can be induced effectively by tumor-cells secreting G-CSF, the stromal cells in tumor can promote tumor growth and metastasis. We further co-injected 4T1 tumor cells (5×10⁵ cells) and gMDSCs (1×10⁷ cells) into the mammary fat pad of test mice. At 18 days post tumor implantation, the primary tumor mass was gently removed surgically. Experimental results showed that the co-transplanted gMDSCs can indeed promote tumor growth and metastasis (FIGS. 2D-E). All gMDSC-eotreated mice were dead at 34 days post tumor resection, whereas 60% of the control set mice were able to sustain as free from, metastasis (FIG. 2E). It is therefore suggested that MDSCs and G-CSF may be aimed as a combination of therapeutic targets for preventing mammary tumor growth and metastasis.

Effect of the Ethanol-extracted Fraction of Bidens pilosa (BP-E) on the Functional and Differentiation Activities of MDSCs and on G-CSF Expression

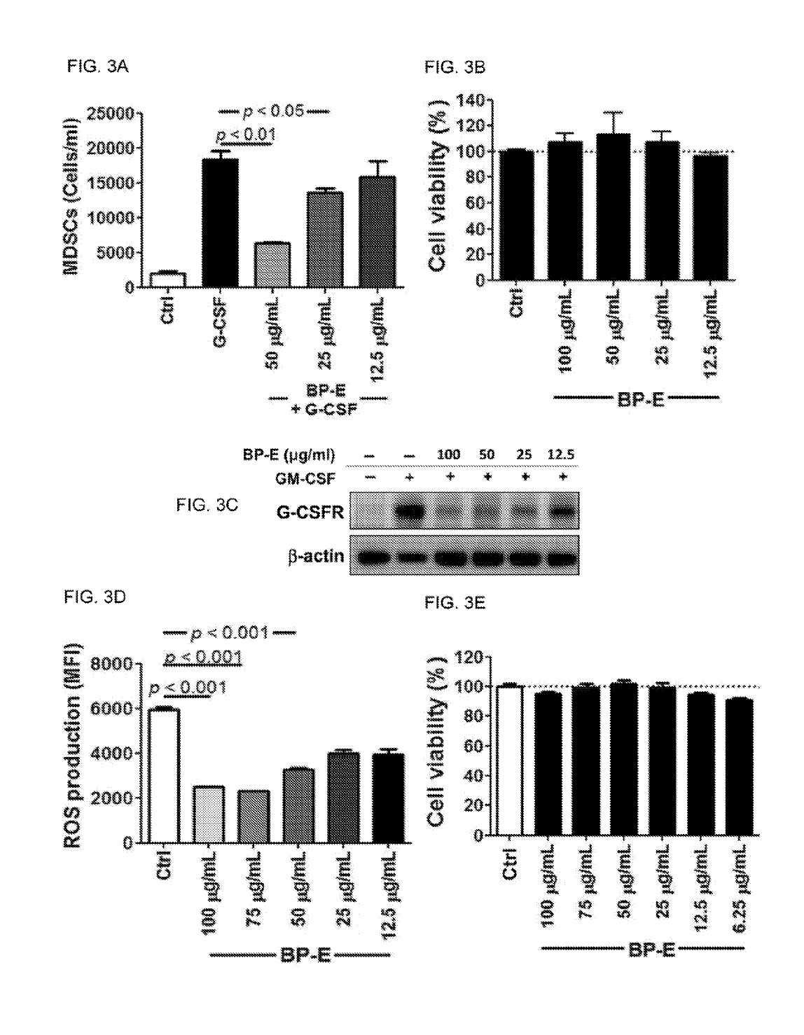

To develop therapeutic agents against tumor metastasis, a number of phyto-extracts or the derived phytochemicals were evaluated for their inhibitory effects on the function and differentiation of MDSCs. It was found that an ethanol partitioned fraction of the Bidens pilosa (BP-E) plant extract significantly suppressed the G-CSF-induced differentiation of gMDSCs from bone marrow cells ex vivo (FIG. 3A). MTT assay showed that BP-E had no significant effect on cell viability of bone marrow cells and the derived MDSCs at a concentration between 100 and 12.5 μg/ml (FIG. 3B). The results of a flow cytometry analysis indicated that BP-E significantly inhibited the production of reactive oxygen species (ROS) in granulocytic MDSCs in a dose dependent manner (FIGS. 3C-D).

Effect of Ethanol-fractionated Phytochemicals from Bidens pilosa (BP-E) on Tumor Metastasis

To evaluate a potential inhibitory effect of oral feeding of BP-E on tumor growth, 4T1-luc2 mouse mammary carcinoma cells were orthotopically implanted into the mammary fat pad of test mice, and subsequently examined in a tumor-resection model. At 7 days post tumor implantation, test mice were divided randomly into BP-E untreated group and treated groups (supplemented via force feeding, an oral dose of 100 mg BP-E/kg body weight/day).

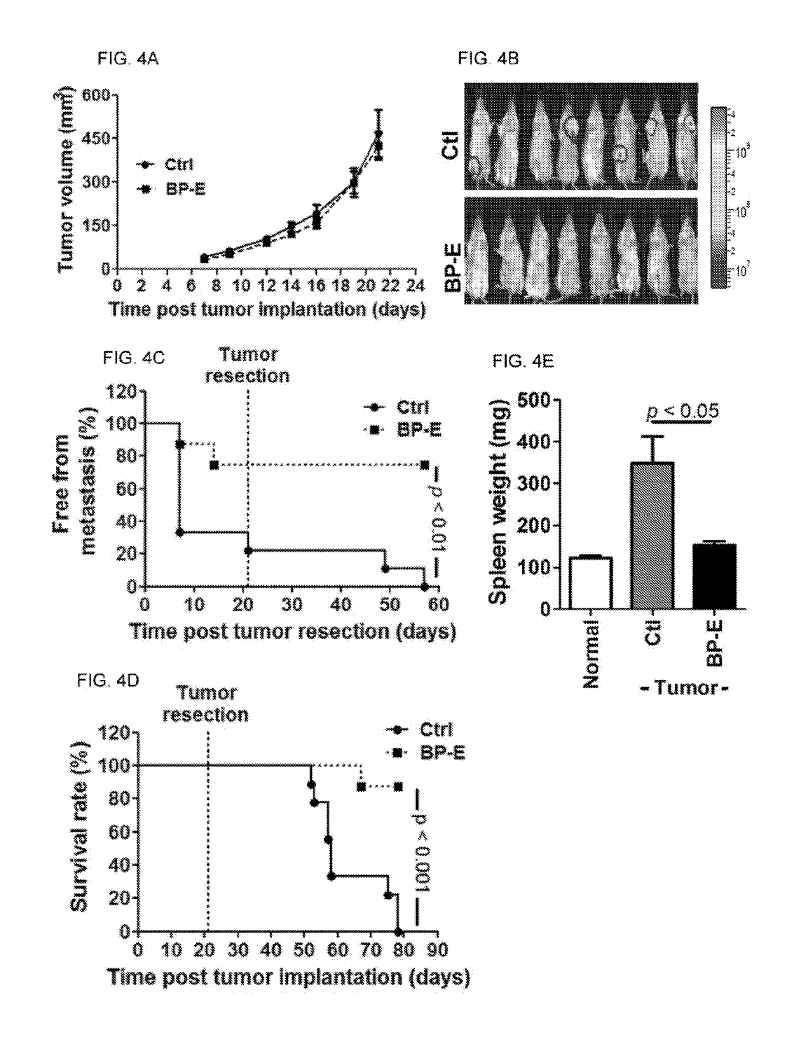

FIG. 4A shows that BP-E had no significant effect on growth of primary tumors, as measured in tumor volume change. We next investigated whether this oral administration of BP-E could confer an effect on tumor metastasis in a tumor resection model. For this experiment, at 21 days post orthotopic tumor implantation, the tissue mass of 4T1-luc2 tumors in test mice was surgically removed. Test animals were then, randomly divided into control (untreated) and BP-E treated groups (100 mg BP-E/kg/day). FIG. 4B shows the result revealed by bioluminescent imaging of the metastatic tumors of each test group, at 7 days post tumor resection. FIG. 4C shows the incidence rate of metastasis for the control group is 62.5% (n=8), whereas the metastasis rate for the BP-E group is only 12.5%. This is a surprisingly drastic difference, and the data is also strongly supported by the sharp contrast in the value of bioluminescent imaging (BLI) seen in FIG. 4B.

It is important to note that in only 7 days, a very short period of time, BP-E feeding was able to effectively suppress tumor metastasis in the tumor resection model. It is also important to point out that the current tumor-resection model was designed to mimic the present human breast cancer patients for treatment following surgery. At 80 days post tumor implantation, the metastasis rate and death rate of the control group mice had reached the level of 100%. The results again strongly suggested that the early onset of the anti-metastatic effect can be successfully maintained tor a prolonged period of time. In contrast, the metastasis rate and the death rate of BP-E treated mice were maintained at 25% and 12.5%, respectively (FIGS. 4C-D).

For subsequent experiment, mice were sacrificed on day 42 post tumor implantation, based on the differentials seen in FIGS. 4C-D, Results seen in FIG. 4E show that 4T1 tumor cells induced a strong splenomegaly activity and BP-E dramatically reduced this tumor-induced splenomegaly (P<0.05) (FIG. 4E).

The myeloid derived suppressor cell (MDSC) populations in spleen tissue of each test group were investigated. Growth of 4T1 tumor strongly induced an accumulation of granulocytic MDSCs in spleen and BP-E effectively reduced (with 50% inhibition) the tumor-induced accumulation of gMDSCs in spleen. In addition, 4T1 tumor cells also slightly increased the monocytic MDSC population in spleen, and BP-E treatment inhibited such an effect in spleen, which indicated that BP-E not only can effectively suppress gMDSC production, but also can inhibit the mMDSC production.

Effect of F1 Fraction of BP-E (BP-E-F1) on ROS Expression in MDSCs and on Differentiation of MDSCs from Bone Marrow Cells

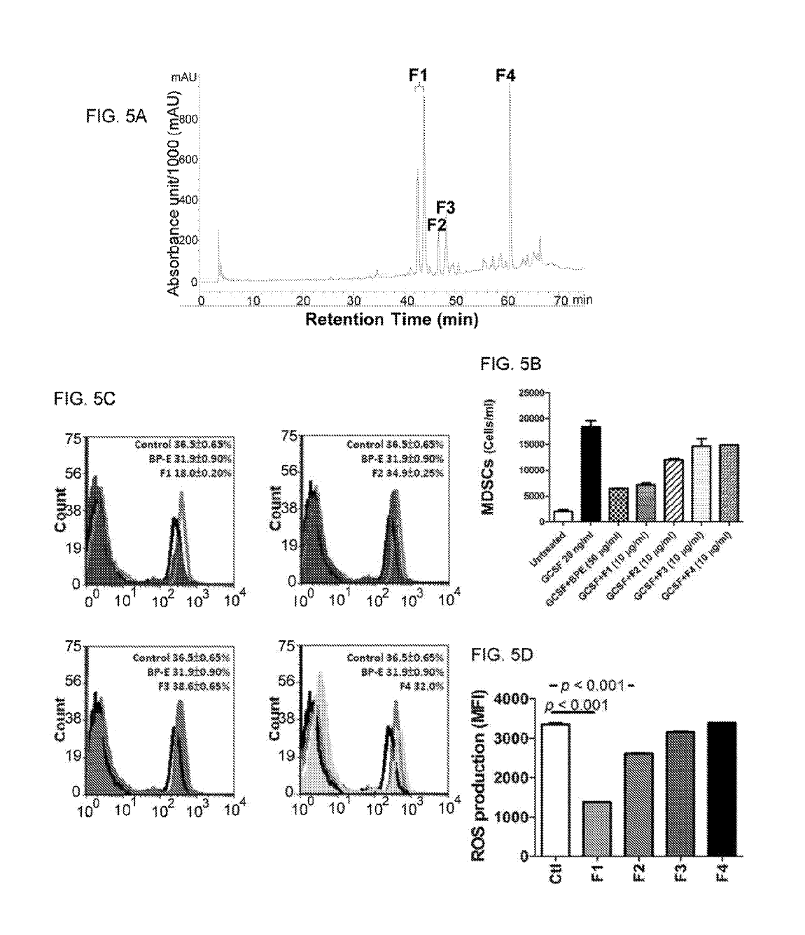

To identify active candidate components or phytochemicals from the BP-E phytoextracts that can confer anti-metastasis activity, BP-E was further fractionationed into 4 sub-fractions (F1 to F4) by using a HPLC analysis with an absorbance of UV at 235 nm (FIG. 5A). These 4 sub-fractions were evaluated for their inhibitory effects on the differentiation and ROS expression of MDSCs under ex vivo culture conditions. FIG. 5B shows that BP-E as well as derived F1 fraction significantly inhibited G-CSF-reduced differentiation of gMDSCs. Furthermore, the F1 fraction also strongly suppressed the ROS expression in gMDSCs (FIGS. 5C and D). These results suggest that the F1 fraction may contain key phytochemical components of BP-E that are responsible for inhibition of the differentiation and function of MDSCs and the resultant anti-tumor metastasis activities.

### Chemical Identification of F1 Phytochemicals

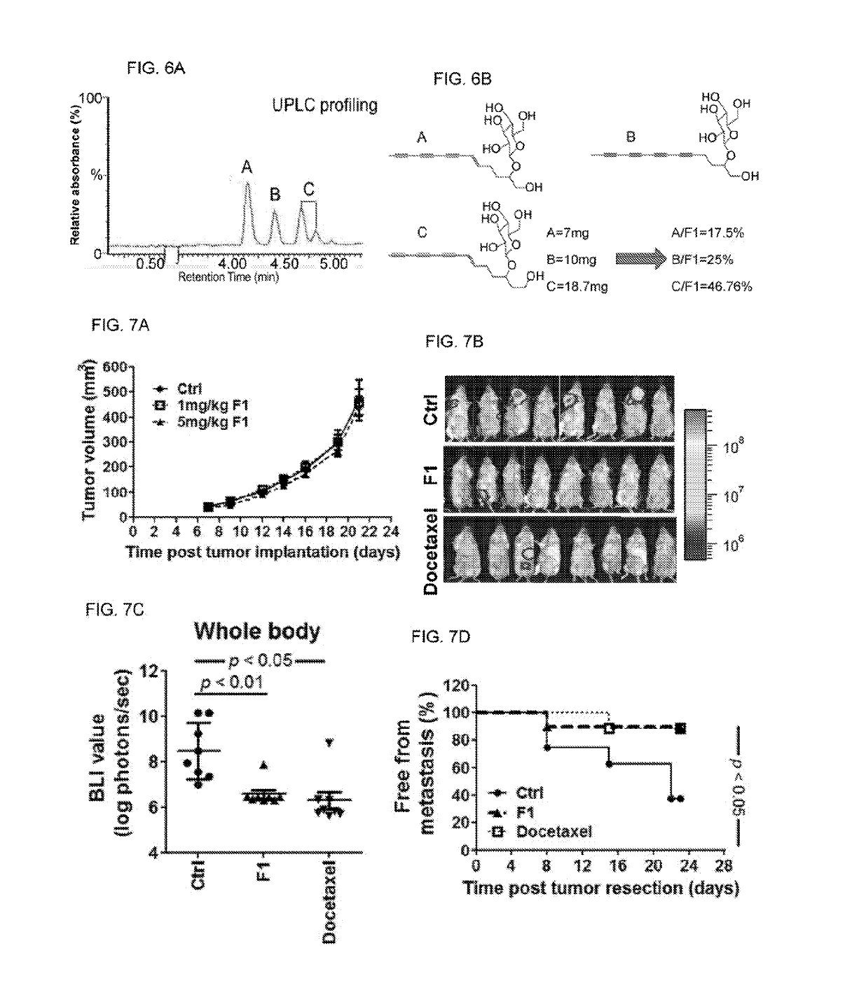

Bio-organic chemical profiling of the F1 fraction phytochemicals was performed by using UPLC, HPLC, NMR and MS/MS assays. F1 fraction was initially chromatographed using a RP-18 UPLC column, and three major compounds (A-C) were isolated (FIG. 6A), and their chemicals structures were subsequently elucidated by spectroscopic methods (FIG. 6B). Compound A, 2-β-D-glucopyranosyloxy-1-hydroxy-5(E)-tridecene-7,9,11-triyne, compound B, 2-D-glucopyranosyloxy-1-hydroxytrideca-5,7,9,11-tetrayne, and compound C, 3-β-D-glucopyranosyloxy-1-hydroxy-6(E)-tetradecene-8,10,12-triyne were comparatively analyzed and confirmed by MS/MS, NMR and our previous studies. The content of compounds A-C in F1 fraction is 89.26% (FIG. 6B).

### Effect of BP-E-F1 on Tumor Metastasis

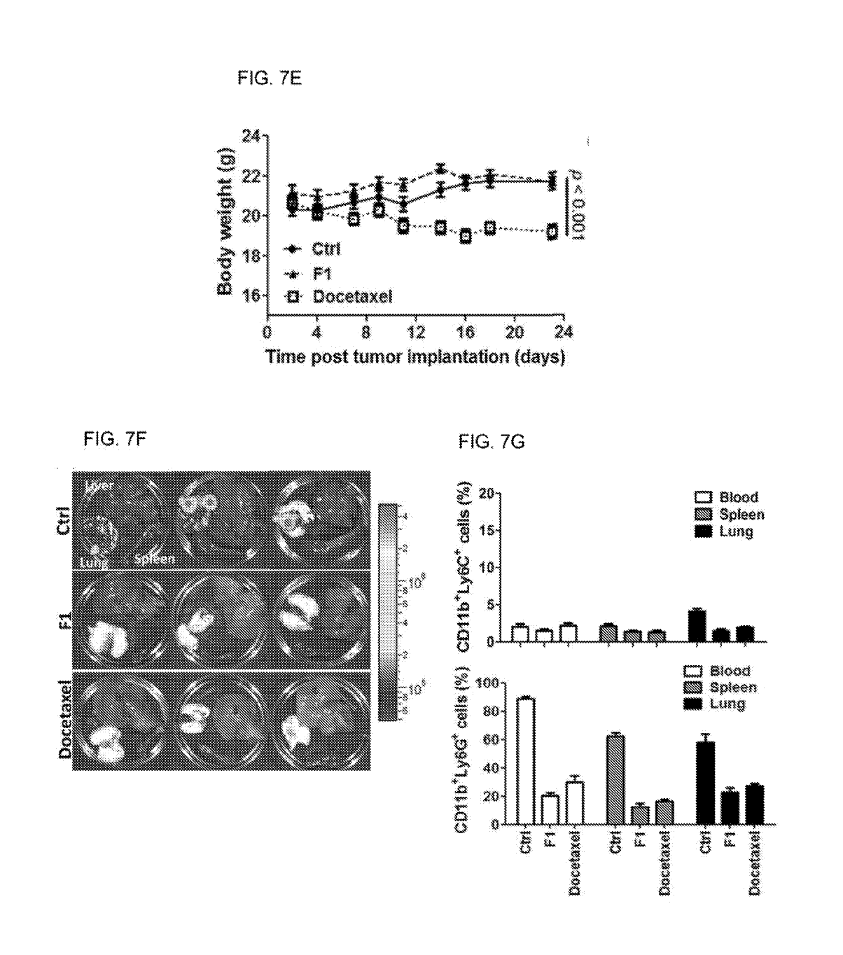

Since we were able to separate the phytochemicals of the BP-E extracts into four major fractions, we investigated possible inhibitory effect of the F1 fraction of BP-E, namely BP-E-F1, on tumor growth in a orthotopic mammary tumor growth/tumor resection mouse model. At 7 days post tumor implantation, test mice were randomly divided into untreated and BP-E-F1 groups (i.e., orally treated with 5 mg BP-E-F1/kg body weight/day). FIG. 7A shows that, like oral, administration of BP-E, BP-E-F1 treatment has little or no significant effect on primary tumor growth, as measured tumor volume.

We investigated the effect of BP-E-F1 on tumor metastasis in the tumor resection model. At 21 days post implantation, the tumor mass was surgically removed gently. Following surgery, each treatment group was randomly divided into control, F1 and the docetaxel groups (i.e., via iv injection with 10 mg docetaxel/kg every other 3 days). FIG. 7B shows the result of bioluminescent imaging of the metastatic tumor for each test group at 23 days post tumor resection. The BLI values for each mouse was quantitatively measured and pooled for each test group. FIG. 7C shows that with virtually an identical pattern, oral administration of BP-E-F1 and the iv-injection of docetaxel were both able to effectively reduce the BLI value observed for test mice. In addition, the metastasis rates determined for the control group F1 and DTX group mice are 62.5%, 12.5% and 12.5%, respectively, at 23 days post tumor resection (FIG. 7D). The body weights of test mice for different treatment groups were found to be distinguishable (FIG. 7E). Unlike treatment with docetaxel, BP-E-F1 treatment not only did not result in body weight loss in test mice, it appeared to have helped the gaining of body weight.

Mice were sacrificed at 23 days post tumor resection, lung, liver and spleen of test mice were excised and tumor metastasis measured by bioluminescent imaging. FIG. 7F shows that lung is the most preferred organ for metastasis of 4T1 tumor cells in test mice, and treatment with BP-E-F1 and docetaxel effectively inhibited tumor metastasis into lung. Treatment with F1 fraction or docetaxel significantly reduced the 4T1 tumor-induced accumulation of granulocytic MDSCs in lung, peripheral blood and spleen of test mice (FIG. 7G).

### BP-E-F1 Inhibits MDSC Activities on Tumor Growth and Metastasis

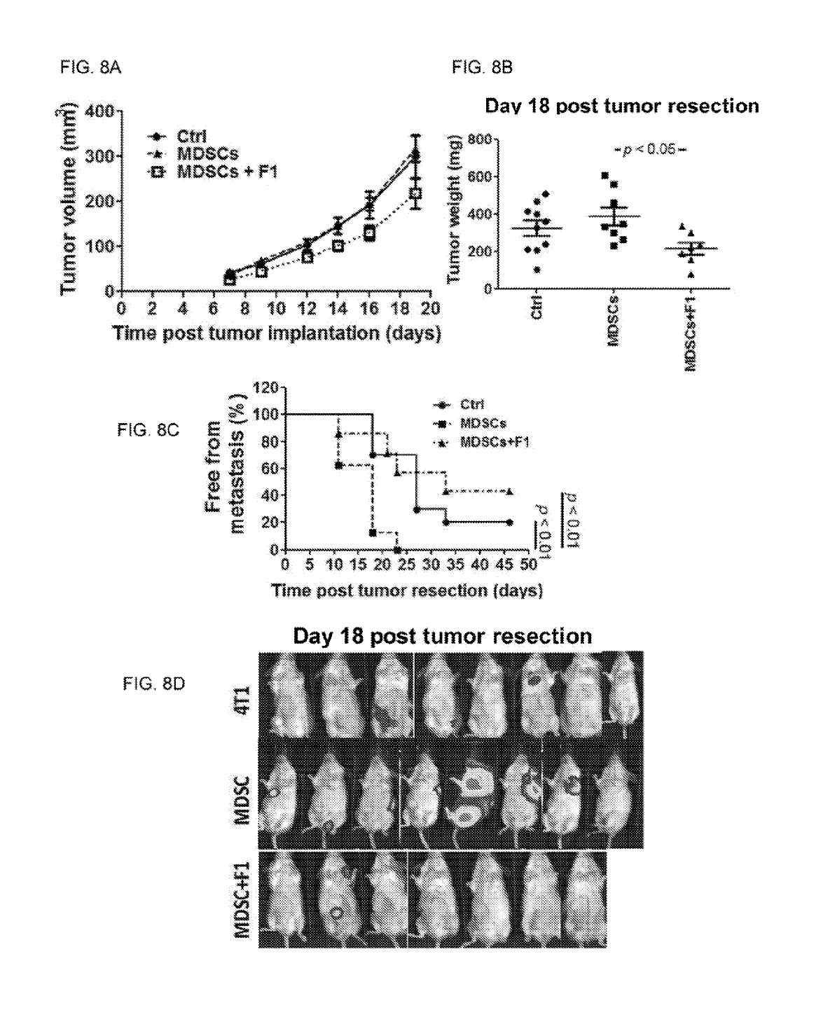

The results suggest that BP-E and its F1 fraction can effectively suppress tumor metastasis via inhibition of differentiation of MDSCs from bone marrow cells and accumulation of MDSCs in the tumor microenvironment. For subsequent experiment, we injected 4T1 cells or co-injected them with granulocytic MDSCs into the mammary fat pad of test mice. At 7 days post tumor implantation, mice were orally fed F1 (5 mg/kg) every day. At 18 days post tumor implantation, the tumor masses of test mice were gently removed surgically and measured. FIGS. 8A-B show that F1 treatment can significantly inhibit the effect of MDSCs on tumor growth as measured weekly by tumor volume and tumor mass (FIGS. 8A-B). In addition, F1 treatment significantly suppressed MDSC-promoted tumor metastasis after tumor resection (FIGS. 8C-D). Our findings suggested that MDSC activity plays a key rote in 4T1 tumor metastasis and can serve as a therapeutic target for lighting against tumor growth and metastasis. BP-E and BP-E-F1 can suppress 4T1 tumor metastasis via inhibition of differentiation of MDSCs from bone marrow cells and accumulation of MDSCs in specific tumor microenvironment.

In summary, we established a murine mammary 4T1-luc2 orthotopic, tumor resection, and subsequent tumor metastasis mouse model. We systemically investigated the roles of MDSCs in tumor growth and metastasis. The findings provide an immunotherapeutic strategy against metastatic cancer that involved high level activities of MDSC differentiation. Granulocytic MDSCs (gMDSCs) are the major MDSC population accumulated in the peripheral blood and spleen tissue of 4T1 tumor-bearing mice, present from early period to later stage of tumor growth (FIG. 1C). The percentage of gMDSCs in present tumor site at day 21 post tumor implantation can be upregulated to 27% (data not shown), and 4T1 tumor cells express consistently high levels of G-CSF, and result in the induction of abundant gMDSC in test mice. Tumor site and spleen tissues are considered to be the key reservoir of MDSCs and their precursors. Due to the massive accumulation of gMDSCs, spleen and tumor site tissues of tumor-bearing mice have become dramatically and rapidly swollen up, as seen at 21 days post tumor implantation (FIG. 1D). These massive increase in gMDSC numbers and their activities may effectively hijack the host immune system, and render it ineffective on inducing antitumor immunities. The role of gMDSCs in promoting tumor growth and metastasis can be further confirmed by our result from the experiment on co-implantation of tumor cells with gMDSCs into the mammary fat pad of test mice, resulting in the gain of a higher tumor weight and higher incidence of metastasis, as compared with an implantation of tumor cells alone (FIGS. 2D-E). MDSCs are clearly shown to play a key role in programming of tumor-induced immunosuppression, facilitating tumor growth and metastasis against host immunity. Therefore, instead of directly targeting and killing tumor cells, effective control and inhibition of MDSC production can be considered and individually modified or monitored for specific patients as a promising strategy for cancer immunotherapy.

Surgery and radiation therapy are current standard treatments tor various cancers, often effective for control at the original tumor of primary tumors site. However, therapies or treatments for metastatic diseases remain to encounter great challenges. Growing body of evidence suggests that chemotherapy, performed as a systemic therapy for metastatic cancers, most often were not able to benefit to cancer patients, instead it often impairs the host immunities, resulting in promotion of tumor growth and spread. We demonstrated that oral administration of the BP-E phytoextract and derived F1 phytochemicals can significantly suppress 4T1 mammary metastasis.

The efficacy of F1 fraction for inhibition of metastasis and MDSC accumulation is at a level just as good as the treatment with docetaxel (FIG. 7). Furthermore, mice fed BP-E-F1 phytochemicals, mainly as three specific polyacetylenes, showed better general health than that of docetaxel-treated mice. Treatment with the polyacetylene phytochemicals, unlike docetaxel, did not result in body weight loss (FIG. 7E) or hair loss in the tested mice. By a direct comparison of the efficacy, ease for drug delivery and cytotoxicity and other side effects of F1 fraction and the currently used clinical drug docetaxel, we suggest that BP-E and the derived F1 fraction of BP-E of polyacetylenes may have high potential in clinical application, as a new generation of anti-cancer agent for use alongside or in combination with existing chemotherapy drugs.

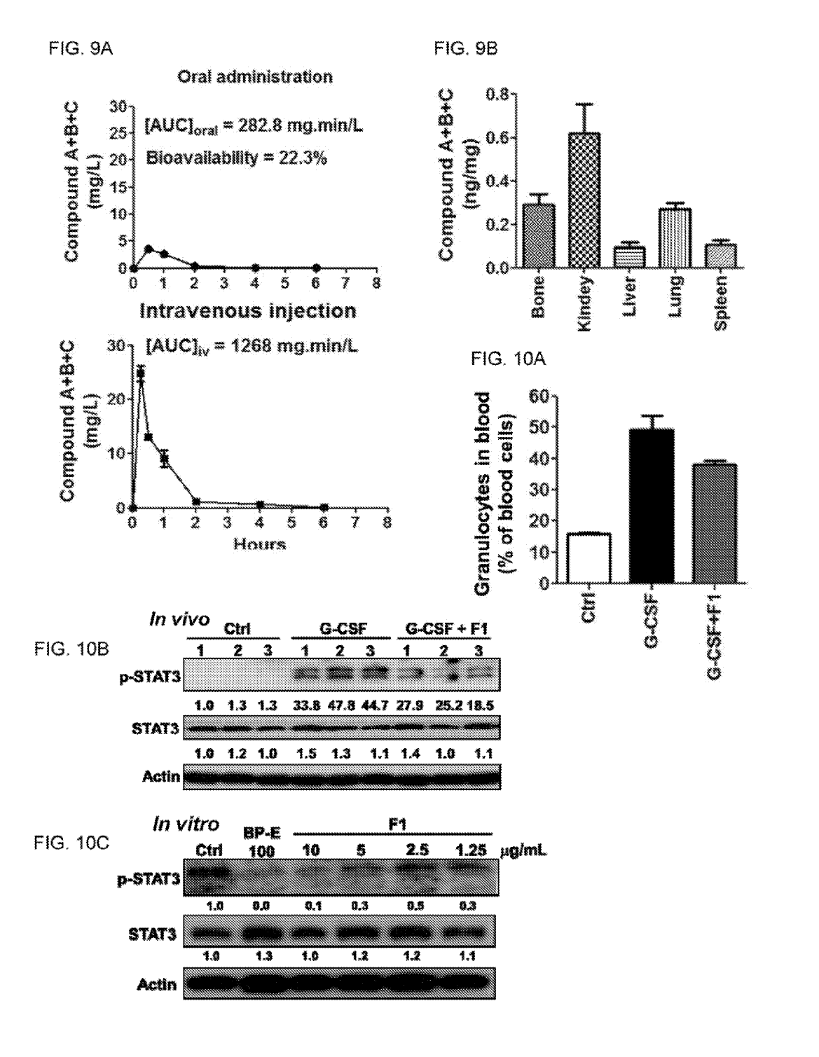

For pharmacological application, the fraction of the administered dose of a test drug that reaches the systemic level in blood circulation is described as bioavailability. We first determined the absolute bioavailability of the three major polyacetylenic glycoside compounds (A, B, C) of BP-E-F1 fraction in blood of test mice. Oral administration was used to investigate the effect of F1 fraction on suppression of 4T1 metastasis. Bioavailability of the three F1 compounds (A-C) was assessed in BALB/c mice (n=12) via administration of F1 fraction by intravenous (iv) or oral delivery, both at 10 mg/kg. The area under curve (AUC) for oral administration and iv administration were experimentally obtained at 282.8 and 1268 mg·min/1, respectively (FIGS. 9A-B). The absolute bioavailability of oral administration can hence be calculated to be 22.3%. We next determined the presence or absence, and the concentration of the three compounds (A-C) in bone, kidney, liver, lung and spleen tissues after the administration of F1 fraction delivered via oral administration. Different organs of test mice (n=3) were collected at 2 hour post oral administration of F1. The concentration of the three compounds (A-C) in different organs was detected (FIG. 10B). This result demonstrate that compounds (A-C) of F1 fraction in serum, kidney, bone, liver, lung and spleen tissues at 20 min to 2 h post oral delivery had maintained at a relatively high concentration in BALB/c mice, indicating that the active phytocompounds of F1 fraction can be readily and directly absorbed into blood circulation and target organs, effecting the suppression of development and function of gMDSCs. The pleasant surprise is that these BP-E/F1 phytochemicals can be more readily bio-available via oral administration.

F1 fraction significantly suppresses the activity in differentiation of MDSCs from bone marrow cells and the functionality of MDSCs, in vitro and in vivo. Tumor-derived G-CSF has been demonstrated to play a key role in promotion of gMDSC development. To investigate the mechanistic role of F1 fraction in inhibition of gMDSC differentiation, we also adopted an approach for the intravenous administration of recombinant G-CSF, aiming to elicit gMDSC activities. Intravenous administration of recombinant G-CSF significantly promotes the percentage of granulocytes in the peripheral blood of test mice, from 16.1% (level in untreated mice) to 49.1% (FIG. 10A). This activity apparently can stimulate the phosphorylation of STAT3, a key transcription factor for differentiation and function of MDSCs, in test bone marrow cells in vivo (FIG. 10B). Oral feeding with F1 can partially suppress the percentage of granulocytes in the peripheral blood of treated mice (FIG. 10A) and effectively reduce the phosphorylation of STAT3 in bone marrow cells of G-CSF-treated mice (FIG. 10B). In an ex vivo experiment, BP-E and F1 treatments also significantly reduced the phosphorylation of STAT3 in in gMDSCs (FIG. 10C). Collectively, The results suggest that BP-E and BP-E-F1 can effectively suppress the differentiation and function of gMDSCs, via inhibiting the tumor-induced activation of STAT3. We suggest that BP-E as well as the BP-E-F1 polyacetylenes from a traditional medicinal plant, Bidens pilosa, may be employed as new category for developing plant natural product-derived immunotherapeutic agents against cancers for use.

1. A method for suppressing, reducing, blocking and/or preventing tumor metastasis in a subject in need thereof, comprising administering to the subject in need thereof a pharmacological composition comprising:
(i) a therapeutically effective amount of Bidens pilosa extract, or more than one polyacetylenic compounds purified or isolated from the B. pilosa extract; and
(ii) a pharmaceutically acceptable carrier.

2. The method of claim 1, wherein the pharmacological composition comprises compounds of formula (I), (II) and (III):

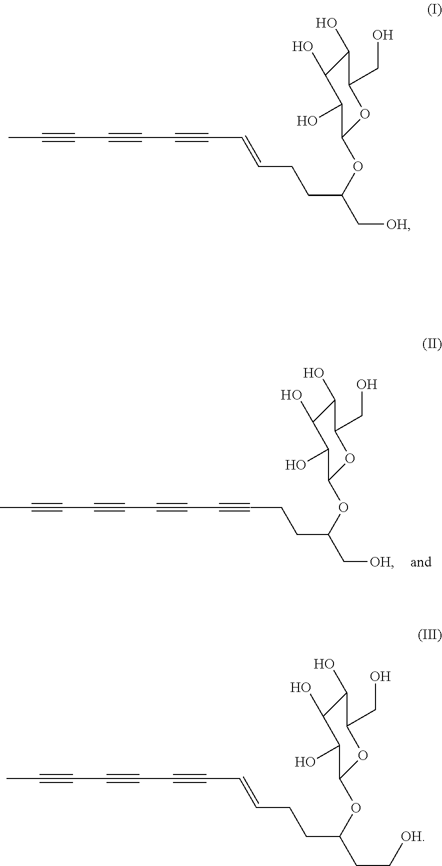 [SMILES: CC#CC#CC#C/C=C/CCC(CO)OC1OC(CO)C(O)C(O)C1O ; CC#CC#CC#CC#CCCC(CO)OC1OC(CO)C(O)C(O)C1O ; "and" ; CC#CC#CC#C/C=C/CCC(CCO)OC1OC(CO)C(O)C(O)C1O]

3. The method of claim 2, wherein the pharmacological composition comprises at least 80% (wt/wt) of compounds 2-β-D-glucopyranosyloxy-1-hydroxy-5(E)-tridecene-7,9,11-triyne, 2-D-glucopyranosyloxy-1-hydroxytrideca-5,7,9,11-tetrayne, and 3-β-D-glucopyranosyloxy-1-hydroxy-6(E)-tetradecene-8,10,12-triyne.

4. The method of claim 2, wherein the pharmacological composition comprises:
(a) 2-β-D-glucopyranosyloxy-1-hydroxy-5(E)-tridecene-7,9,11 -triyne,
(b) 2-D-glucopyranosyloxy-1-hydroxytrideca-5,7,9,11-tetrayne, and
(c) 3-β-D-glucopyranosyloxy-1-hydroxy-6(E)-tetradecene-8,10,12-triyne at a ratio ranging from 1:1:1 to 1:2:4.

5. The method of claim 1, wherein the subject has breast cancer, or is a post-operative cancer surgery patient.

6. The method of claim 1, wherein the amount of the Bidens pilosa extract or the more than one polyacetylenic compounds purified or isolated from the B. pilosa extract is effective in inhibiting differentiation, functional activities, and population of granulocytic myeloid-derived suppressor cells (gMDSCs) and suppressing tumor metastasis without causing cytotoxicity or apoptosis to the gMDSCs.

7. The method, of claim 1, wherein the pharmaceutical composition is in a dosage form selected from the group consisting of oral, intravenous, intramuscular, and subcutaneous.

8. The method of claim 1, wherein the amount of the Bidens pilosa extract or the more than one polyacetylenic compounds purified or isolated from the B. pilosa extract is effective in inhibiting tumor metastasis into lung, and accumulation of granulocytic MDSCs in lung, peripheral blood and spleen of the subject in need thereof.

9. The method of claim 1, wherein the Bidens pilosa extract is;
(i) an ethanol extract of B. pilosa; or
(ii) a first fraction eluted from an HPLC column loaded with a mixture containing the ethanol extract of B. pilosa; or
(iii) a repeatedly re-chromatographed fraction of the ethanol extract of B. pilosa.

10. The method of claim 9, wherein the B. pilosa extract comprises no less than 89% (w/w) of polyacetylenic compounds.

11. The method of claim 9, wherein the pharmaceutical composition comprises a human equivalent dose of:
(a) 10-1000 mg of the ethanol extract of B. pilosa/Kg body weight×(0.025 Kg/human body weight in Kg)^{0.33}, or
(b) 0.5-1000 mg of the first fraction/Kg body weight×(0.025 Kg/human body weight in Kg)^{0.33}.

12. A method for inhibiting differentiation, functional activities, and population of granulocytic myeloid-derived supressor cells (gMDSCs) and/or suppressing metastatic cancer in a subject in need thereof, comprising administering to the subject in need thereof a pharmacological composition comprising:
(i) a therapeutically effective amount of Bidens pilosa extract, or more than one polyacetylenic compounds purified or isolated from the B. pilosa extract; and
(ii) a pharmaceutically acceptable carrier.

13. The method of claim 12, wherein the pharmacological composition comprises compounds of formula (I), (II) and (III):

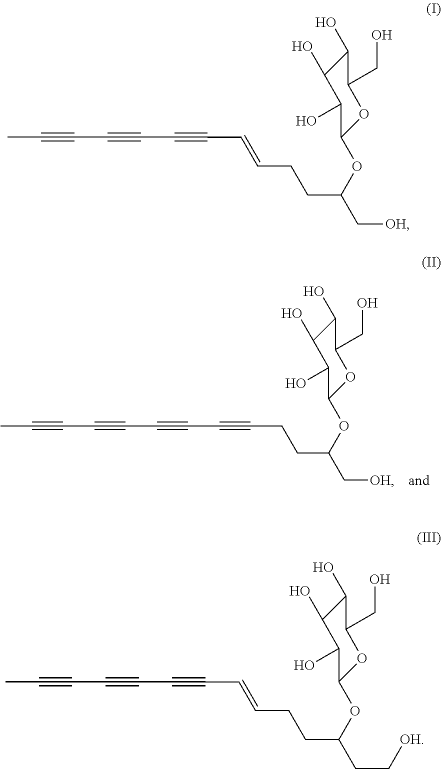 [SMILES: CC#CC#CC#C/C=C/CCC(CO)OC1OC(CO)C(O)C(O)C1O ; CC#CC#CC#CC#CCCC(CO)OC1OC(CO)C(O)C(O)C1O ; "and" ; CC#CC#CC#C/C=C/CCC(CCO)OC1OC(CO)C(O)C(O)C1O]

14. The method of claim 12, wherein the subject has breast cancer, or is a post-operative cancer surgery patient, or in need for control, blockage and prevention of cancer metastasis.

15. The method of claim 12, wherein the pharmacological composition comprises at least 80% (wt/wt) of compounds 2-β-D-glucopyranosyloxy-1-hydroxy-5(E)-tridecene-7,9,11-triyne, 2-D-glucopyranosyloxy-1-hydroxytrideca-5,7,9,11-tetrayne, and 3-β-D-glucopyransyloxy-1-hydroxy-6(E)-tetradecene-8,10,12-triyne.

16. The method of claim 1, wherein the Bidens pilosa extract, or the polyacetylenic compounds purified or isolated from the B. pilosa extract suppress, reduce, block, and/or prevent tumor metastasis via inhibiting the differentiation and function of gMDSC.
